(19) United States
(12) Patent Application Publication
(10) Pub. No.: US 2024/0173346 A1
(43) Pub. Date: May 30, 2024

(54) LINE-1 INHIBITORS TO TREAT CNS AND SYSTEMIC DISEASES

(71) Applicant: TRANSPOSON THERAPEUTICS, INC., San Diego, CA (US)

(72) Inventors: Eckard WEBER, Los Angeles, CA (US); Michael G. CORDINGLEY, Québec (CA)

(21) Appl. No.: 18/550,498

(22) PCT Filed: Mar. 15, 2022
(86) PCT No.: PCT/US2022/020362

§ 371 Date: Sep. 14, 2023

Related U.S. Application Data
(60) Provisional application No. 63/161,050, filed on Mar. 15, 2021.

(51) Int. Cl. A61K 31/7076; A61K 31/7072

(52) U.S. Cl. CPC A61K 31/7076; A61K 31/7072

(57) ABSTRACT

The present disclosure provides methods of treating or preventing a CNS or systemic disease in a subject in need thereof comprising administering to the subject a therapeutically effective amount of a LINE-1 inhibitor, or a pharmaceutical composition thereof.

### BACKGROUND OF THE INVENTION

### Field of the Invention

The present disclosure provides methods of treating or preventing central nervous system (CNS) and systemic diseases in a subject comprising administering a LINE-1 inhibitor, or a pharmaceutical composition thereof, to the subject.

### Background

Long INterspersed Element-1 (LINE-1 or L1) retrotransposons form the only autonomously active family of transposable elements in humans. They are expressed and mobile in the germline, in embryonic stem cells, and in the early embryo, but are silenced in most somatic tissues. LINE-1 plays an important role in individual genome variations through insertional mutagenesis and sequence transduction, which occasionally lead to genetic diseases and disorders. The LINE-1 element codes for two proteins, ORF1p and ORF2p, which are essential for its mobility. ORF1p is an RNA-binding protein with nucleic acid chaperone activity. ORF2p possesses endonuclease and reverse transcriptase activities. These proteins and the LINE-1 RNA assemble into a ribonucleoprotein particle (LINE-1 RNP)—the core of the retrotransposition machinery. The LINE-1 RNP mediates the synthesis of new LINE-1 copies upon cleavage of the target DNA and reverse transcription of the LINE-1 RNA at the target site. The LINE-1 element takes benefit of cellular host factors to complete its life cycle, however several cellular pathways also limit the cellular accumulation of LINE-1 RNPs and their deleterious activities. See, e.g., Pizarro and Cristofari (2016) Front. Cell Dev. Biol. 4:14. doi: 10.3389/fcell.2016.00014. There exists a need for new methods to treat or prevent CNS or systemic diseases in subjects.

### BRIEF SUMMARY OF THE INVENTION

In one aspect, the present disclosure provides methods of treating or preventing a CNS disease, e.g., ataxia-telangiectasia, or a systemic disease, e.g., age-related macular degeneration, in a subject in need thereof with a therapeutically effective amount of a LINE-1 inhibitor. Exemplary LINE-1 inhibitors include, but are not limited to, islatravir, censavudine, and elvucitabine.

In another aspect, the present disclosure provides methods of treating or preventing a CNS disease or systemic disease in a subject in need thereof with a therapeutically effective amount of a LINE-1 inhibitor in combination with a therapeutically effective amount of one or more optional therapeutic agents.

In another aspect, the present disclosure provides a kit comprising a LINE-1 inhibitor for treating or preventing a CNS disease or systemic disease.

In another aspect, the present disclosure provides a LINE-1 inhibitor for use in treating or preventing a CNS disease or systemic disease in a subject in need thereof.

In another aspect, the present disclosure provides the use of a LINE-1 inhibitor for the manufacture of a medicament for treating or preventing a CNS disease or systemic disease in a subject.

In another aspect, the present disclosure provides a LINE-1 inhibitor of Table 1.

In another aspect, the present disclosure provides a LINE-1 inhibitor of Table 2.

### DETAILED DESCRIPTION OF THE INVENTION

Applicant unexpectedly discovers that a LINE-1 inhibitor, e.g., islatravir, censavudine, or elvucitabine, can be used to treat certain CNS diseases and systemic diseases. The present disclosure thus provides methods to treat or prevent, e.g., ataxia-telangiectasia, age-related macular degeneration, systemic lupus erythematosus, IFN-associated autoimmune disease, e.g., psoriasis, Fanconi Anemia, idiopathic pulmonary fibrosis, or cardiovascular disease, e.g., coronary heart disease, in a subject.

### I. Therapeutic Methods and Uses

A LINE-1 inhibitor, or pharmaceutical composition thereof, can be administered to a subject in need thereof, e.g., a subject already suffering from a CNS or systemic disease, a subject suspected of having a CNS or systemic disease, or a subject at risk of acquiring a CNS or systemic disease.

In one embodiment, the disclosure provides a method of treating or preventing a CNS disease in a subject in need thereof, the method comprising administering a therapeutically effective amount of a LINE-1 inhibitor to the subject.

In another embodiment, the disclosure provides a method of treating or preventing a systemic disease in a subject in need thereof, the method comprising administering a therapeutically effective amount of a LINE-1 inhibitor to the subject.

In another embodiment, the disclosure provides a LINE-1 inhibitor, or a composition thereof, for use in treating a CNS disease.

In another embodiment, the disclosure provides a LINE-1 inhibitor, or a composition thereof, for use in treating a systemic disease.

In another embodiment, the disclosure provides the use of a LINE-1 inhibitor in the manufacture of a medicament for treating a CNS disease.

In another embodiment, the disclosure provides the use of a LINE-1 inhibitor in the manufacture of a medicament for treating a systemic disease.

In another embodiment, the CNS disease is ataxia-telangiectasia.

In another embodiment, the systemic disease is age-related macular degeneration, systemic lupus erythematosus, IFN-associated autoimmune disease, e.g., psoriasis, Fanconi Anemia, idiopathic pulmonary fibrosis, or cardiovascular disease. In another embodiment, the systemic disease is age-related macular degeneration. In another embodiment, the systemic disease is systemic lupus erythematosus. In another embodiment, the systemic disease is an IFN-associated autoimmune disease, e.g., psoriasis. In another embodiment, the systemic disease is Fanconi Anemia. In another embodiment, the systemic disease is idiopathic pulmonary fibrosis. In another embodiment, the systemic disease is cardiovascular disease.

In another embodiment, the LINE-1 inhibitor is islatravir, censavudine, or elvucitabine.

In another embodiment, the LINE-1 inhibitor is islatravir.

In another embodiment, the LINE-1 inhibitor is censavudine.

In another embodiment, the LINE-1 inhibitor is elvucitabine.

In another embodiment, the LINE-1 inhibitor is a compound of Formula I, a compound of Formula II, or a compound of Formula III. See below.

In another embodiment, the LINE-1 inhibitor is a compound of Table 1. See below.

In another embodiment, the LINE-1 inhibitor is a compound of Table 2. See below.

In another embodiment, the subject is (a) not infected with the HIV virus, (b) is not suspected of being infected with the HIV virus, and/or (c) is not being treated to prevent infection with the HIV virus.

In one embodiment, LINE-1 inhibitor is administered to a subject having a CNS or systemic disease as a single agent.

In another embodiment, LINE-1 inhibitor is administered to a subject having a CNS or systemic disease in combination with one or more optional therapeutic agents.

In another embodiment, LINE-1 inhibitor is administered to a subject having a CNS or systemic disease in combination with one optional therapeutic agent.

In another embodiment, LINE-1 inhibitor is administered to a subject having a CNS or systemic disease in combination with two optional therapeutic agents.

In another embodiment, LINE-1 inhibitor is administered to a subject having a CNS or systemic disease in combination with three optional therapeutic agents.

The LINE-1 inhibitor and the one or more optional therapeutic agents can be administered in combination under one or more of the following conditions: at different periodicities, at different durations, at different concentrations, by different administration routes, etc.

In one embodiment, the LINE-1 inhibitor and the one or more optional therapeutic agents are administered in combination to a subject as part of a single pharmaceutical composition.

In another embodiment, the LINE-1 inhibitor and the one or more optional therapeutic agents are administered in combination to a subject separately, e.g., as two or more separate pharmaceutical compositions. In this case, two separate pharmaceutical compositions, e.g., one comprising the LINE-1 inhibitor an optional therapeutic agent, are administered to a subject. The separate pharmaceutical compositions can be administered to the subject, for example, at different periodicities, at different durations, or by different administration routes, e.g., the LINE-1 inhibitor can be administered orally and the optional therapeutic agent(s) can be administered intravenously.

In another embodiments, the LINE-1 inhibitor is administered to the subject prior to the one or more optional therapeutic agents, e.g., 0.5, 1, 2, 3, 4, 5, 10, 12, or 18 hours, 1, 2, 3, 4, 5, or 6 days, or 1, 2, 3, or 4 weeks prior to the administration of the one or more optional therapeutic agents.

In another embodiments, the LINE-1 inhibitor is administered to the subject after the one or more optional therapeutic agents, e.g., 0.5, 1, 2, 3, 4, 5, 10, 12, or 18 hours, 1, 2, 3, 4, 5, or 6 days, or 1, 2, 3, or 4 weeks after the administration of the one or more optional therapeutic agents.

In another embodiments, the LINE-1 inhibitor and the one or more optional therapeutic agents are administered concurrently.

In one embodiment, the LINE-1 inhibitor is administered to the subject according to a continuous dosing schedule.

In one embodiment, the LINE-1 inhibitor is administered to the subject according to an intermittent dosing schedule.

In one embodiment, the LINE-1 inhibitor is orally administered to the subject.

The therapeutic methods provided herein comprise administering a LINE-1 inhibitor to a subject having a CNS or systemic disease in an amount which is effective to achieve its intended purpose. While individual needs vary, determination of optimal ranges of effective amounts of each component is within the skill of the art. Typically, the LINE-1 inhibitor is administered in an amount from about 0.01 mg/kg to about 500 mg/kg, about 0.05 mg/kg to about 100 mg/kg, about 0.05 mg/kg to about 50 mg/kg, or about 0.05 mg/kg to about 10 mg/kg. In one embodiment, the LINE-1 inhibitor is administered once a day. In another embodiment, LINE-1 inhibitor is administered twice a day. In one embodiment, LINE-1 inhibitor is administered three times a day. In one embodiment, LINE-1 inhibitor is administered four times a day. These dosages are exemplary, but there can be individual instances in which higher or lower dosages are merited, and such are within the scope of this disclosure. In practice, the physician determines the actual dosing regimen that is most suitable for an individual subject, which can vary with the age, weight, and response of the particular subject.

The unit dose may comprise from about 0.01 mg to about 1000 mg, e.g., about 1 mg to about 500 mg, e.g., about 1 mg to about 250 mg, e.g., about 1 mg to about 100 mg of the LINE-1 inhibitor. For example, the unit oral dose of LINE-1 inhibitor may comprise, for example 1 mg, 2 mg, 3 mg, 4 mg, 5 mg, 6 mg, 7 mg, 8 mg, 9 mg, or 10 mg of islatravir. The unit dose may be administered one or more times daily, e.g., as one or more tablets or capsules. The unit dose may also be administered by any suitable route, e.g., orally, by IV, inhalation or subcutaneously to the subject. In practice, the physician determines the actual dosing regimen that is most suitable for an individual subject, which can vary with the age, weight, and response of the particular subject.

In one embodiment, the LINE-1 inhibitor is administered to a subject in an amount from about 0.1 mg to about 100 mg once a day, twice a day, three times a day, or four times a day. In another embodiment, the LINE-1 inhibitor is administered to a subject in an amount from about 1 mg to about 50 mg per day.

In one embodiment, the LINE-1 inhibitor is administered to the subject in a single dose. In another embodiment, the LINE-1 inhibitor is administered to the subject in two divided doses. In another embodiment, the LINE-1 inhibitor is administered to the subject in three divided doses. In another embodiment, the LINE-1 inhibitor is administered to the subject in four divided doses.

The LINE-1 inhibitor can be administered to a subject in the form of a raw chemical or as part of a pharmaceutical composition containing the LINE-1 inhibitor combined with a suitable pharmaceutically acceptable carrier. Such a carrier can be selected from pharmaceutically acceptable excipients, vehicles, and auxiliaries. The term “pharmaceutically acceptable carrier,” “pharmaceutically acceptable vehicle,” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, PA, 19th ed. 1995.

A pharmaceutical composition comprising the LINE-1 inhibitor can contain from about 0.01 to 99 percent by weight, e.g., from about 0.25 to 75 percent by weight, of the LINE-1 inhibitor, e.g., about 1%, about 5%, about 10%, about 15%, about 20%, about 25%, about 30%, about 35%, about 40%, about 45%, about 50%, about 55%, about 60%, about 65%, about 70%, or about 75% by weight of the LINE-1 inhibitor.

The LINE-1 inhibitor, or pharmaceutical composition comprising the LINE-1 inhibitor, can be administered by any suitable route, for example by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration to a subject. Dosage forms depend on the route administration. Dosage forms include, but are not limited to, tablets, dragees, slow release lozenges, capsules, liquid solutions, liquid suspensions, oral/nasal spray, transdermal patch, thin dissolvable film, ointments, sustained or controlled release implants, mouth rinses and mouth washes, gels, hair rinses, hair gels, and shampoos, and suppositories, as well as suitable solutions for administration by intravenous infusion, and suitable suspensions for administration subcutaneous injection, and suitable powders for reconstitution. Parenteral administration can be accomplished using a needle and syringe or using other technique known in the art. In one embodiment, the LINE-1 inhibitor is administered orally to the subject. In one embodiment, the LINE-1 inhibitor is administered subcutaneously to the subject. In one embodiment, the LINE-1 inhibitor is administered intravenously to the subject.

The LINE-1 inhibitor and pharmaceutical compositions comprising the LINE-1 inhibitor may be administered to any subject which may experience the beneficial effects of inhibitor of LINE-1. The term “subject” as used herein refers to any human or animal that is in need of or might benefit from therapy. Foremost among such subjects are mammals, e.g., humans, although the methods and compositions provided herein are not intended to be so limited. Other subjects include veterinary animals, e.g., cows, sheep, pigs, horses, dogs, cats and the like. In one embodiment, the subject is a human. In one embodiment, the subject is an animal.

The pharmaceutical preparations provided herein are manufactured by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries can be suitable flow-regulating agents and lubricants. Suitable auxiliaries include, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of LINE-1 inhibitors may be administered to a subject. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers and other additives.

Therapeutically effective amounts of a LINE-1 inhibitor formulated in accordance with standard pharmaceutical practices are administered to a subject in need thereof. Whether such a treatment is indicated depends on the individual case and is subject to medical assessment (diagnosis) that takes into consideration signs, symptoms, and/or malfunctions that are present, the risks of developing particular signs, symptoms and/or malfunctions, and other factors.

Pharmaceutical compositions include those wherein a LINE-1 inhibitor is administered in an effective amount to achieve its intended purpose. The exact formulation, route of administration, and dosage is determined by an individual physician in view of the diagnosed condition or disease. Dosage amount and interval can be adjusted individually to provide levels of the LINE-1 inhibitor that is sufficient to maintain therapeutic effects.

Toxicity and therapeutic efficacy of the LINE-1 inhibitor can be determined by standard pharmaceutical procedures in cell cultures or experimental animals, e.g., for determining the maximum tolerated dose (MTD) of a compound, which defines as the highest dose that causes no toxicity in a subject. The dose ratio between the maximum tolerated dose and therapeutic effects is the therapeutic index. The dosage can vary within this range depending upon the dosage form employed, and the route of administration utilized. Determination of a therapeutically effective amount is well within the capability of those skilled in the art, especially in light of the detailed disclosure provided herein.

A therapeutically effective amount of the LINE-1 inhibitor required for use in therapy varies with the nature of the disease being treated, the length of time that activity is desired, and the age and the condition of the subject, and ultimately is determined by the attendant physician. For example, dosage amounts and intervals can be adjusted individually to provide plasma levels of a LINE-1 inhibitor that are sufficient to maintain the desired therapeutic effects. The desired dose conveniently can be administered in a single dose, or as multiple doses administered at appropriate intervals, for example as one, two, three, four or more subdoses per day.

### II. Kits

In another embodiment, the present disclosure provides kits comprising a LINE-1 inhibitor, or a composition comprising LINE-1 inhibitor, packaged in a manner that facilitates their use to practice methods of the present disclosure.

In one embodiment, the kit includes a LINE-1 inhibitor, or a composition thereof, packaged in a container, such as a sealed bottle or vessel, with a label affixed to the container or included in the kit that describes use of the compound or composition to practice the method of the disclosure. In one embodiment, the compound or composition is packaged in a unit dosage form. The kit may include a single dose or multiple doses of a LINE-1 inhibitor, or a pharmaceutical composition thereof.

In another embodiment, the kit includes LINE-1 inhibitor, or a composition thereof, and one or more optional therapeutic agents, or a composition thereof.

### III. Definitions

The term “LINE-1 inhibitor” and the like as used herein refers to a compound that inhibits human LINE-1 retrotransposition. In one embodiment, the LINE-1 inhibitor is a compound that inhibits human LINE-1 retrotransposition with a half maximal inhibitory concentration (IC₅₀) of 1 μM or less in a HeLa cell-based dual-luciferase assay as described in EXAMPLE 1, see below. See also Jones et al., (2008) PLoS ONE 3 (2): e1547. doi:10.1371/journal.pone.0001547; Xie et al., (2011) Nucleic Acids Res. 39 (3): e16. doi: 10.1093/nar/gkq1076. In another embodiment, the IC₅₀ is 0.5 μM or less. In another embodiment, the IC₅₀ is 0.25 μM or less. In another embodiment, the IC₅₀ is 0.15 μM or less. In another embodiment, the IC₅₀ is 0.1 μM or less. In another embodiment, the IC₅₀ is 0.05 μM or less. In another embodiment, the IC₅₀ is 0.01 μM or less. In another embodiment, the IC₅₀ is 0.005 μM or less. LINE-1 inhibitors are described, for example, in WO 2020/154656.

In one embodiment, the LINE-1 inhibitor is islatravir. Islatravir is a compound having the following chemical structure:

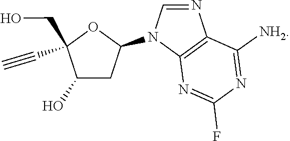 [SMILES: C#C[C@]1(CO)O[C@@H](n2cnc3c(N)nc(F)nc32)C[C@@H]1O]

Islatravir (also known as EDdA, MK-8591 or 2′-deoxy-4′-ethynyl-2-fluoroadenosine) and its method of synthesis is described in U.S. Pat. No. 7,625,877.

In some embodiments, islatravir is administered to a subject daily in an amount that ranges from about 0.1 mg to about 20 mg, e.g., from about 0.5 mg to about 15 mg, e.g., from about 1 mg to about 10 mg. In some embodiments, about 0.1 mg, about 0.15 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 1 mg, about 2 mg, about 3 mg, about 4 mg, about 5 mg, about 6 mg, about 7 mg, about 8 mg, about 9 mg, or about 10 mg of islatravir is administered to the subject per day.

In some embodiments, islatravir is administered monthly to a subject in an amount that ranges from about 50 to about 150 mg. In some embodiments, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of islatravir is administered to the subject per month.

In some embodiments, islatravir is administered to a subject by continuous release from an implant. In some embodiments, the implant comprises about 30 to about 80 mg islatravir. In some embodiments, the implant comprises about 30 mg, about 35 mg, about 40 mg, about 45 mg, about 50 mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 70 mg, about 75 mg, or about 80 mg. In some embodiments, islatravir is administered to a subject by continuous release from an implant containing about 54 mg or about 62 mg islatravir.

In some embodiments, islatravir is administered to a patient is (a) not infected with the HIV virus, (b) is not suspected of being infected with the HIV virus, and/or (c) is not being treated to prevent infection with the HIV virus.

In one embodiment, the LINE-1 inhibitor is censavudine. Censavudine is a compound having the following chemical structure:

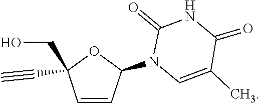 [SMILES: C#C[C@@]1(CO)C=C[C@H](n2cc(C)c(=O)[nH]c2=O)O1]

Censavudine (also known as 4′-Ed4T, 4′-ethynyl-d4T, 4′-ethynylstavudine, BMS-986001, OBP-601, festinavir) and its method of synthesis is described in U.S. Pat. No. 7,589,078.

In one embodiment, the LINE-1 inhibitor is elvucitabine. Elvucitabine is a compound having the following chemical structure:

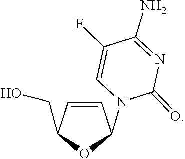 [SMILES: Nc1nc(=O)n([C@@H]2C=C[C@H](CO)O2)cc1F]

Elvucitabine and its method of synthesis is described in U.S. Pat. No. 5,627,160.

In another embodiment, the LINE-1 inhibitor is censavudine, islatravir, lamivudine (3TC), zidovudine (AZT), tenofovir, tenofovir disoproxil, tenofovir alafenamide, stavudine (d4T), didanosine (ddI), emtricitabine (FTC), entecavir (ETV), 2′,3′-dideoxyguanosine (ddG), 2′,3′-dideoxyadenosine (ddA), 2′-fluoro-2′,3′-dideoxyarabinosyladenine (F-ddA), efavirenz (EFV), nevirapine (NVP), abacavir (ABC), adefovir dipivoxil, or telbivudine.

In another embodiment, the LINE-1 inhibitor is a compound of Formula I:

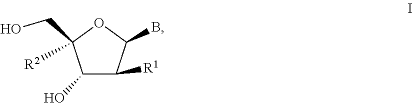

or a pharmaceutically acceptable salt or solvate thereof, or a tautomer thereof, wherein:

B is selected from the group consisting of:

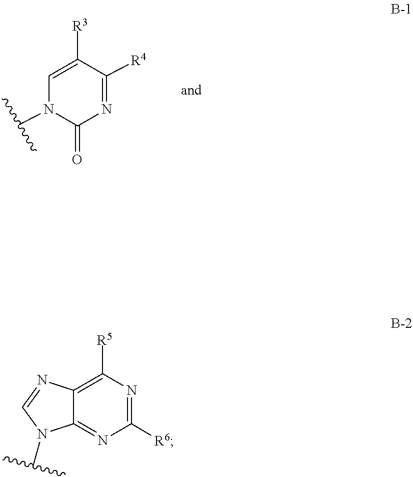

R¹ is selected from the group consisting of hydrogen and —OH;

R² is selected from the group consisting of methyl, ethynyl, and —CN;

R³ is selected from the group consisting of hydrogen, fluoro, chloro, bromo, iodo and methyl;

R⁴ is selected from the group consisting of —NH₂ and —OH;

R⁵ is selected from the group consisting of —NH₂ and —OH; and

R⁶ is selected from the group consisting of hydrogen, fluoro, chloro, and —NH₂

In another embodiment, the LINE-1 inhibitor is a compound of Formula II:

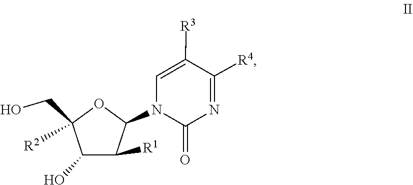

or a pharmaceutically acceptable salt or solvate thereof, or a tautomer thereof, wherein R¹, R², R³, and R⁴ are as defined in connection with Formula I.

In another embodiment, the LINE-1 inhibitor is a compound of Formula II, wherein R³ is hydrogen.

In another embodiment, the LINE-1 inhibitor is a compound of Formula II, wherein R³ is selected from the group consisting of fluoro and chloro.

In another embodiment, the LINE-1 inhibitor is a compound of Formula II, wherein R³ is methyl.

In another embodiment, the LINE-1 inhibitor is a compound of Formula II, wherein R⁴ is —NH₂.

In another embodiment, the LINE-1 inhibitor is a compound of Formula II, wherein R⁴ is —OH.

In another embodiment, the LINE-1 inhibitor is a compound of Formula III:

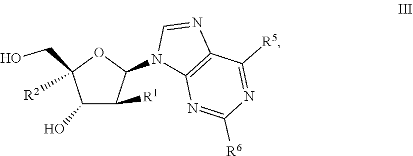

or a pharmaceutically acceptable salt or solvate thereof, or a tautomer thereof, wherein R¹, R², R⁵, and R⁶ are as defined in connection with Formula I.

In another embodiment, the LINE-1 inhibitor is a compound of Formula III, wherein R⁵ is —NH₂.

In another embodiment, the LINE-1 inhibitor is a compound of Formula III, wherein R⁵ is —OH.

In another embodiment, the LINE-1 inhibitor is a compound of Formula III, wherein R⁶ is hydrogen.

In another embodiment, the LINE-1 inhibitor is a compound of Formula III, wherein R⁶ is chloro.

In another embodiment, the LINE-1 inhibitor is a compound of Formula III, wherein R⁶ is fluoro.

In another embodiment, the LINE-1 inhibitor is a compound of Formula III, wherein R⁶ is —NH₂.

In another embodiment, the LINE-1 inhibitor is a compound of any one of Formulae I-III, wherein R¹ is hydrogen.

In another embodiment, the LINE-1 inhibitor is a compound of any one of Formulae I-III, wherein R¹ is —OH.

In another embodiment, the LINE-1 inhibitor is a compound of any one of Formulae I-III, wherein R² is methyl.

In another embodiment, the LINE-1 inhibitor is a compound of any one of Formulae I-III, wherein R² is ethynyl.

In another embodiment, the LINE-1 inhibitor is a compound of any one of Formulae I-III, wherein R² is —CN.

In another embodiment, the LINE-1 inhibitor is a compound of Table 1:

| Cpd. No. | Structure |
|---|---|
| 1 | 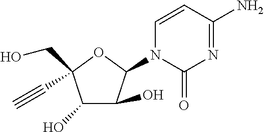 [SMILES: C#C[C@]1(CO)O[C@@H](n2ccc(N)nc2=O)[C@@H](O)[C@@H]1O] |
| 2 | 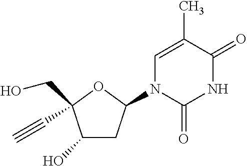 [SMILES: C#C[C@]1(CO)O[C@@H](n2cc(C)c(=O)[nH]c2=O)C[C@@H]1O] |
| 3 | 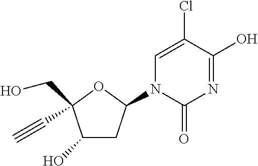 [SMILES: C#C[C@]1(CO)O[C@@H](n2cc(Cl)c(O)nc2=O)C[C@@H]1O] |
| 4 | 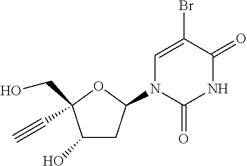 [SMILES: C#C[C@]1(CO)O[C@@H](n2cc(Br)c(=O)[nH]c2=O)C[C@@H]1O] |
| 5 | 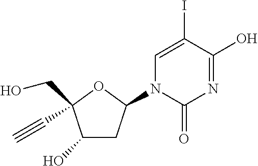 [SMILES: C#C[C@]1(CO)O[C@@H](n2cc(I)c(O)nc2=O)C[C@@H]1O] |
| 6 | 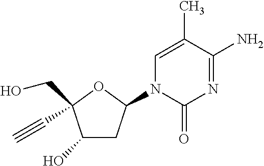 [SMILES: C#C[C@]1(CO)O[C@@H](n2cc(C)c(N)nc2=O)C[C@@H]1O] |
| 7 | 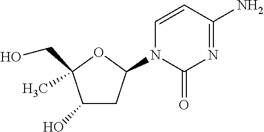 [SMILES: C[C@]1(CO)O[C@@H](n2ccc(N)nc2=O)C[C@@H]1O] |
| 8 | 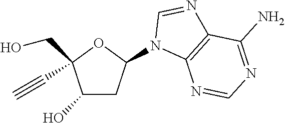 [SMILES: C#C[C@]1(CO)O[C@@H](n2cnc3c(N)ncnc32)C[C@@H]1O] |
| 9 | 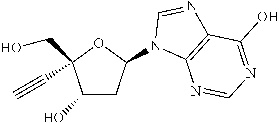 [SMILES: C#C[C@]1(CO)O[C@@H](n2cnc3c(O)ncnc32)C[C@@H]1O] |
| 10 | 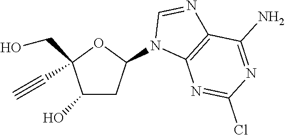 [SMILES: C#C[C@]1(CO)O[C@@H](n2cnc3c(N)nc(Cl)nc32)C[C@@H]1O] |
| 11 | 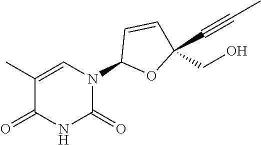 [SMILES: CC#C[C@]1(CO)C=C[C@H](n2cc(C)c(=O)[nH]c2=O)O1] |
| 12 | 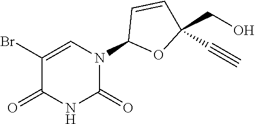 [SMILES: C#C[C@@]1(CO)C=C[C@H](n2cc(Br)c(=O)[nH]c2=O)O1] |
| 13 | 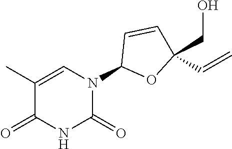 [SMILES: C=C[C@@]1(CO)C=C[C@H](n2cc(C)c(=O)[nH]c2=O)O1] |
| 14 | 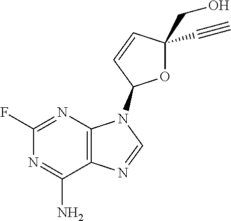 [SMILES: C#C[C@@]1(CO)C=C[C@H](n2cnc3c(N)nc(F)nc32)O1] |
| 15 | 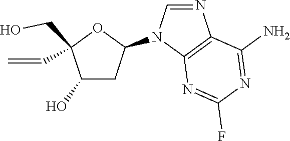 [SMILES: C=C[C@]1(CO)O[C@@H](n2cnc3c(N)nc(F)nc32)C[C@@H]1O] |
| 16 | 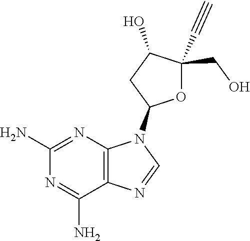 [SMILES: C#C[C@]1(CO)O[C@@H](n2cnc3c(N)nc(N)nc32)C[C@@H]1O] |
| 17 | 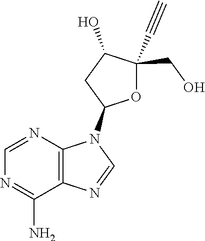 [SMILES: C#C[C@]1(CO)O[C@@H](n2cnc3c(N)ncnc32)C[C@@H]1O] |
| 18 | 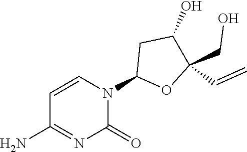 [SMILES: C=C[C@]1(CO)O[C@@H](n2ccc(N)nc2=O)C[C@@H]1O] |
| 19 | 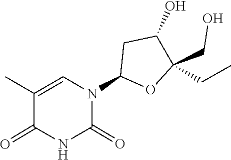 [SMILES: CC[C@]1(CO)O[C@@H](n2cc(C)c(=O)[nH]c2=O)C[C@@H]1O] |
| 20 | 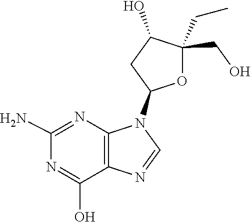 [SMILES: CC[C@]1(CO)O[C@@H](n2cnc3c(O)nc(N)nc32)C[C@@H]1O] |

or a pharmaceutically acceptable salt or solvate thereof, or a tautomer thereof.

In another embodiment, the LINE-1 inhibitor is a compound of Table 2:

TABLE 2

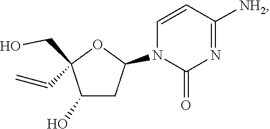 [SMILES: C=C[C@]1(CO)O[C@@H](n2ccc(N)nc2=O)C[C@@H]1O]

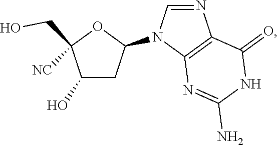 [SMILES: N#C[C@]1(CO)O[C@@H](n2cnc3c(=O)[nH]c(N)nc32)C[C@@H]1O]

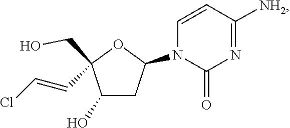 [SMILES: Nc1ccn([C@H]2C[C@H](O)[C@@](/C=C/Cl)(CO)O2)c(=O)n1]

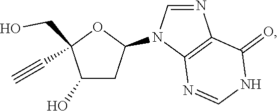 [SMILES: C#C[C@]1(CO)O[C@@H](n2cnc3c(=O)[nH]cnc32)C[C@@H]1O]

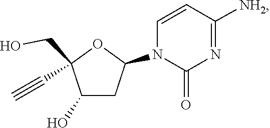 [SMILES: C#C[C@]1(CO)O[C@@H](n2ccc(N)nc2=O)C[C@@H]1O]

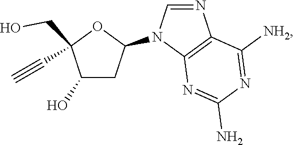 [SMILES: C#C[C@]1(CO)O[C@@H](n2cnc3c(N)nc(N)nc32)C[C@@H]1O]

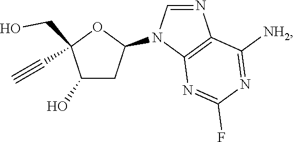 [SMILES: C#C[C@]1(CO)O[C@@H](n2cnc3c(N)nc(F)nc32)C[C@@H]1O]

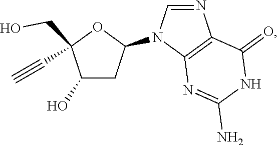 [SMILES: C#C[C@]1(CO)O[C@@H](n2cnc3c(=O)[nH]c(N)nc32)C[C@@H]1O]

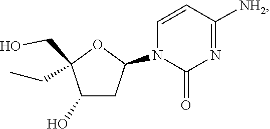 [SMILES: CC[C@]1(CO)O[C@@H](n2ccc(N)nc2=O)C[C@@H]1O]

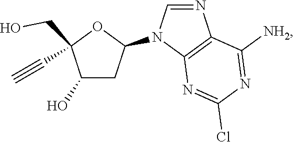 [SMILES: C#C[C@]1(CO)O[C@@H](n2cnc3c(N)nc(Cl)nc32)C[C@@H]1O]

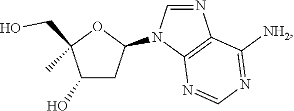 [SMILES: C[C@]1(CO)O[C@@H](n2cnc3c(N)ncnc32)C[C@@H]1O]

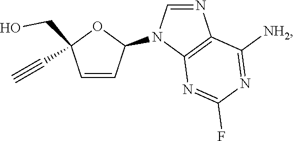 [SMILES: C#C[C@@]1(CO)C=C[C@H](n2cnc3c(N)nc(F)nc32)O1]

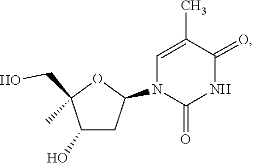 [SMILES: Cc1cn([C@H]2C[C@H](O)[C@@](C)(CO)O2)c(=O)[nH]c1=O]

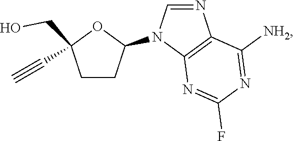 [SMILES: C#C[C@@]1(CO)CC[C@H](n2cnc3c(N)nc(F)nc32)O1]

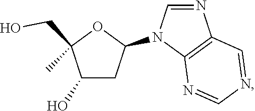 [SMILES: C[C@]1(CO)O[C@@H](n2cnc3cncnc32)C[C@@H]1O]

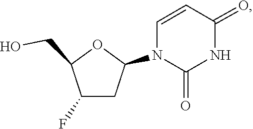 [SMILES: O=c1ccn([C@H]2C[C@H](F)[C@@H](CO)O2)c(=O)[nH]1]

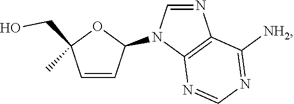 [SMILES: C[C@@]1(CO)C=C[C@H](n2cnc3c(N)ncnc32)O1]

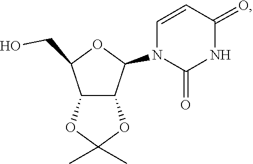 [SMILES: CC1(C)O[C@@H]2[C@H](O1)[C@@H](CO)O[C@H]2n1ccc(=O)[nH]c1=O]

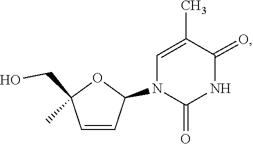 [SMILES: Cc1cn([C@H]2C=C[C@@](C)(CO)O2)c(=O)[nH]c1=O]

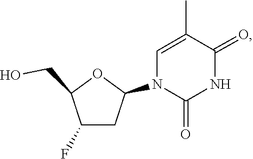 [SMILES: Cc1cn([C@H]2C[C@H](F)[C@@H](CO)O2)c(=O)[nH]c1=O]

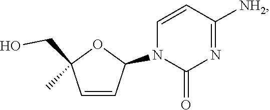 [SMILES: C[C@@]1(CO)C=C[C@H](n2ccc(N)nc2=O)O1]

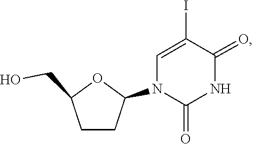 [SMILES: O=c1[nH]c(=O)n([C@H]2CC[C@@H](CO)O2)cc1I]

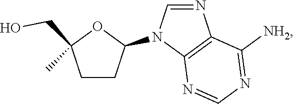 [SMILES: C[C@@]1(CO)CC[C@H](n2cnc3c(N)ncnc32)O1]

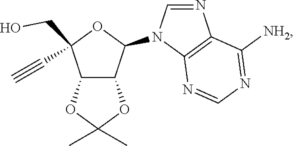 [SMILES: C#C[C@]1(CO)O[C@@H](n2cnc3c(N)ncnc32)[C@@H]2OC(C)(C)O[C@@H]21]

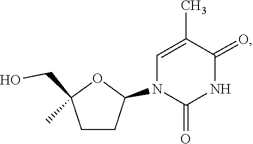 [SMILES: Cc1cn([C@H]2CC[C@@](C)(CO)O2)c(=O)[nH]c1=O]

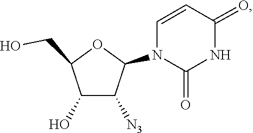 [SMILES: [N-]=[N+]=N[C@@H]1[C@H](O)[C@@H](CO)O[C@H]1n1ccc(=O)[nH]c1=O]

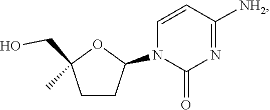 [SMILES: C[C@@]1(CO)CC[C@H](n2ccc(N)nc2=O)O1]

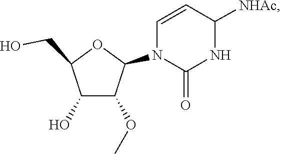 [SMILES: CO[C@@H]1[C@H](O)[C@@H](CO)O[C@H]1N1C=CC(NC(C)=O)NC1=O]

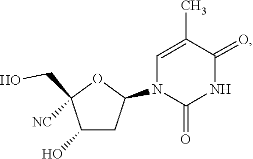 [SMILES: Cc1cn([C@H]2C[C@H](O)[C@@](C#N)(CO)O2)c(=O)[nH]c1=O]

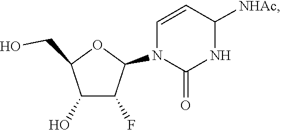 [SMILES: CC(=O)NC1C=CN([C@@H]2O[C@H](CO)[C@@H](O)[C@H]2F)C(=O)N1]

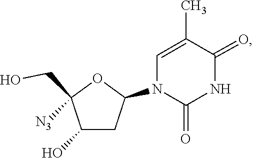 [SMILES: Cc1cn([C@H]2C[C@H](O)[C@](CO)(N=[N+]=[N-])O2)c(=O)[nH]c1=O]

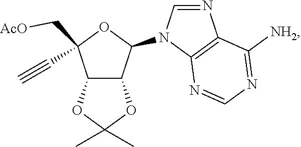 [SMILES: C#C[C@]1(COC(C)=O)O[C@@H](n2cnc3c(N)ncnc32)[C@@H]2OC(C)(C)O[C@@H]21]

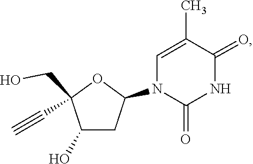 [SMILES: C#C[C@]1(CO)O[C@@H](n2cc(C)c(=O)[nH]c2=O)C[C@@H]1O]

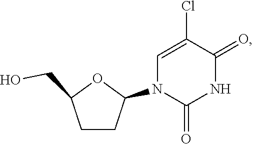 [SMILES: O=c1[nH]c(=O)n([C@H]2CC[C@@H](CO)O2)cc1Cl]

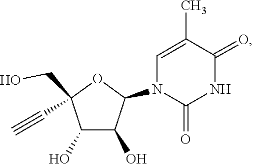 [SMILES: C#C[C@]1(CO)O[C@@H](n2cc(C)c(=O)[nH]c2=O)[C@@H](O)[C@@H]1O]

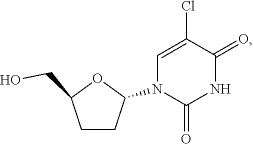 [SMILES: O=c1[nH]c(=O)n([C@@H]2CC[C@@H](CO)O2)cc1Cl]

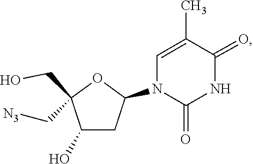 [SMILES: Cc1cn([C@H]2C[C@H](O)[C@](CO)(CN=[N+]=[N-])O2)c(=O)[nH]c1=O]

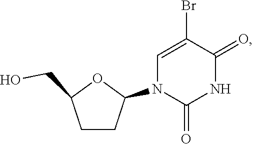 [SMILES: O=c1[nH]c(=O)n([C@H]2CC[C@@H](CO)O2)cc1Br]

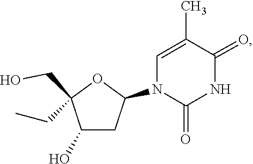 [SMILES: CC[C@]1(CO)O[C@@H](n2cc(C)c(=O)[nH]c2=O)C[C@@H]1O]

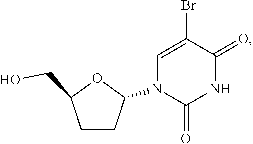 [SMILES: O=c1[nH]c(=O)n([C@@H]2CC[C@@H](CO)O2)cc1Br]

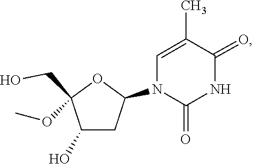 [SMILES: CO[C@]1(CO)O[C@@H](n2cc(C)c(=O)[nH]c2=O)C[C@@H]1O]

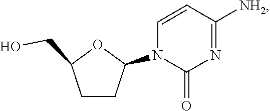 [SMILES: Nc1ccn([C@H]2CC[C@@H](CO)O2)c(=O)n1]

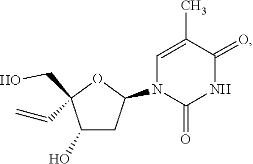 [SMILES: C=C[C@]1(CO)O[C@@H](n2cc(C)c(=O)[nH]c2=O)C[C@@H]1O]

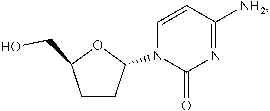 [SMILES: Nc1ccn([C@@H]2CC[C@@H](CO)O2)c(=O)n1]

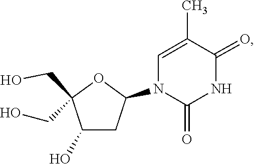

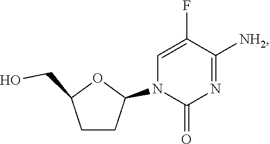 [SMILES: Nc1nc(=O)n([C@H]2CC[C@@H](CO)O2)cc1F]

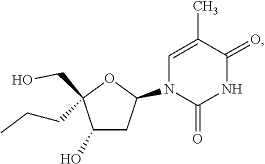 [SMILES: CCC[C@]1(CO)O[C@@H](n2cc(C)c(=O)[nH]c2=O)C[C@@H]1O]

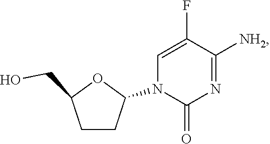 [SMILES: Nc1nc(=O)n([C@@H]2CC[C@@H](CO)O2)cc1F]

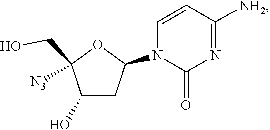 [SMILES: [N-]=[N+]=N[C@]1(CO)O[C@@H](n2ccc(N)nc2=O)C[C@@H]1O]

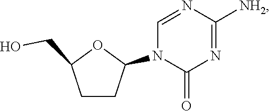 [SMILES: Nc1ncn([C@H]2CC[C@@H](CO)O2)c(=O)n1]

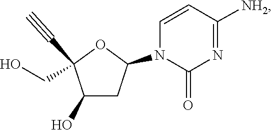 [SMILES: C#C[C@@]1(CO)O[C@@H](n2ccc(N)nc2=O)C[C@H]1O]

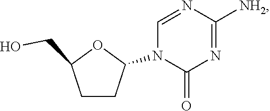 [SMILES: Nc1ncn([C@@H]2CC[C@@H](CO)O2)c(=O)n1]

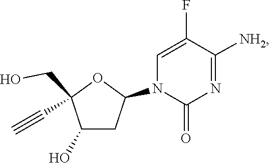 [SMILES: C#C[C@]1(CO)O[C@@H](n2cc(F)c(N)nc2=O)C[C@@H]1O]

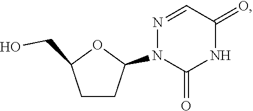 [SMILES: O=c1cnn([C@H]2CC[C@@H](CO)O2)c(=O)[nH]1]

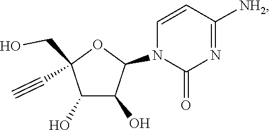 [SMILES: C#C[C@]1(CO)O[C@@H](n2ccc(N)nc2=O)[C@@H](O)[C@@H]1O]

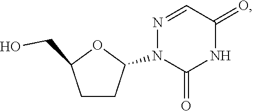 [SMILES: O=c1cnn([C@@H]2CC[C@@H](CO)O2)c(=O)[nH]1]

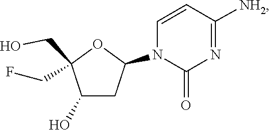 [SMILES: Nc1ccn([C@H]2C[C@H](O)[C@](CO)(CF)O2)c(=O)n1]

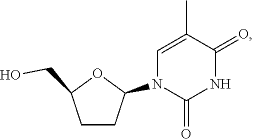 [SMILES: Cc1cn([C@H]2CC[C@@H](CO)O2)c(=O)[nH]c1=O]

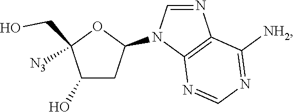 [SMILES: [N-]=[N+]=N[C@]1(CO)O[C@@H](n2cnc3c(N)ncnc32)C[C@@H]1O]

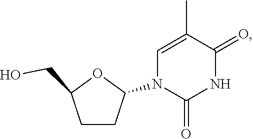 [SMILES: Cc1cn([C@@H]2CC[C@@H](CO)O2)c(=O)[nH]c1=O]

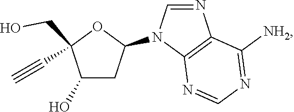 [SMILES: C#C[C@]1(CO)O[C@@H](n2cnc3c(N)ncnc32)C[C@@H]1O]

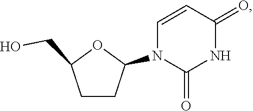 [SMILES: O=c1ccn([C@H]2CC[C@@H](CO)O2)c(=O)[nH]1]

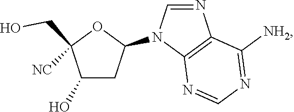 [SMILES: N#C[C@]1(CO)O[C@@H](n2cnc3c(N)ncnc32)C[C@@H]1O]

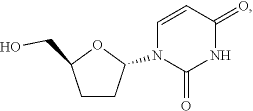 [SMILES: O=c1ccn([C@@H]2CC[C@@H](CO)O2)c(=O)[nH]1]

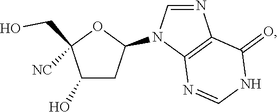 [SMILES: N#C[C@]1(CO)O[C@@H](n2cnc3c(=O)[nH]cnc32)C[C@@H]1O]

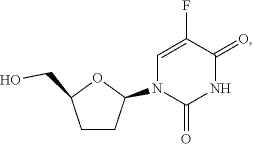 [SMILES: O=c1[nH]c(=O)n([C@H]2CC[C@@H](CO)O2)cc1F]

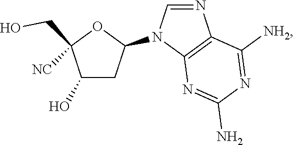 [SMILES: N#C[C@]1(CO)O[C@@H](n2cnc3c(N)nc(N)nc32)C[C@@H]1O]

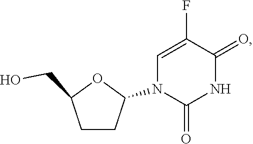 [SMILES: O=c1[nH]c(=O)n([C@@H]2CC[C@@H](CO)O2)cc1F]

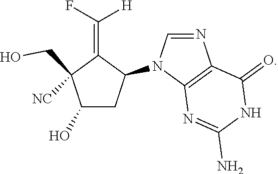 [SMILES: N#C[C@]1(CO)/C(=C\F)[C@@H](n2cnc3c(=O)[nH]c(N)nc32)C[C@@H]1O]

or a pharmaceutically acceptable salt or solvate thereof, or a tautomer thereof.

The compounds of Tables 1 and 2 may be synthesized, for example, as described in Nomura et al., J. Med. Chem. 42:2901-2908 (1999); Ohrui, H., Proc. Jpn. Acad Ser. B 87:53-65 (2011); Banuelos-Sanchez et al., Cell Chemical Biology 26:1095-1109 (2019); Kirby et al., Antimicrobial Agents and Chemotherapy 57:6254-6264 (2013), or JP Patent No. 6767011.

The terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language, e.g., “such as,” provided herein, is intended to better illustrate the disclosure and is not a limitation on the scope of the disclosure unless otherwise claimed. No language in the specification should be construed as indicating any non-claimed element as essential to the practice of the disclosure.

The term “about,” as used herein, includes the recited number ±10%. Thus, “about 10” means 9 to 11.

As used herein, the terms “treat,” “treating,” “treatment,” and the like refer to eliminating, reducing, or ameliorating a CNS or systemic disease and/or symptoms associated therewith. Although not precluded, treating the CNS or systemic disease does not require that the disease or symptoms associated therewith be completely eliminated. However, in one embodiment, administration of a LINE-1 inhibitor leads to complete elimination of the disease and associated symptoms.

As used herein, the terms “prevent,” “preventing,” “prevention” and the like refer to a method of preventing the onset of a CNS or systemic disease and/or symptoms associated therewith, or barring a subject from acquiring the infectious disease. The terms “prevent,” “preventing,” and “prevention” also include delaying the onset of a CNS or systemic disease and/or its attendant symptoms, and reducing a subject's risk of acquiring the infectious disease. The terms “prevent,” “preventing” and “prevention” includes “prophylactic treatment,” which refers to reducing the probability of redeveloping a CNS or systemic disease, or of a recurrence of a previously-controlled a CNS or systemic disease in a subject who does not have, but is at risk of or is susceptible to, redeveloping the disease or a recurrence of the disease.

The term “therapeutically effective amount,” as used herein, refers to that amount of a LINE-1 inhibitor and/or one or more optional therapeutic agents sufficient to result in amelioration of one or more symptoms of a disease, or prevent advancement of a disease, or cause regression of the disease. For example, with respect to the treatment of a CNS or systemic disease, in one embodiment, a therapeutically effective amount will refer to the amount of a therapeutic agent that causes a therapeutic response.

The term “container” means any receptacle and closure therefore suitable for storing, shipping, dispensing, and/or handling the LINE-1 inhibitor. Non-limiting exemplary containers include vials, ampules, bottles, and syringes.

The term “insert” means information accompanying a pharmaceutical product that provides a description of how to administer the product, along with the safety and efficacy data required to allow the physician, pharmacist, and subject to make an informed decision regarding use of the product. The package insert generally is regarded as the “label” for a pharmaceutical product.

In some embodiments, when administered in combination, two or more therapeutic agents can have a synergistic effect. The terms “synergy,” “synergistic,” “synergistically” and derivations thereof, such as in a “synergistic effect” or a “synergistic combination” or a “synergistic composition” as used herein refer to circumstances under which the biological activity of a combination of an agent and at least one additional therapeutic agent is greater than the sum of the biological activities of the respective agents when administered individually. For example, the term “synergistically effective” as used herein refers to the interaction between a LINE-1 inhibitor and another therapeutic agent that causes the total effect of the drugs to be greater than the sum of the individual effects of each drug. Berenbaum, Pharmacological Reviews 41:93-141 (1989).

Synergy can be expressed in terms of a “Synergy Index (SI),” which generally can be determined by the method described by F. C. Kull et al. Applied Microbiology 9, 538 (1961), from the ratio determined by:

QₐQ_{A}+Q_{b}Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

The terms “intermittent dose administration,” “intermittent dosing schedule,” and similar terms as used herein refer to, i.e., not continuous, administration, of a LINE-1 inhibitor to a subject.

Intermittent dose administration of a LINE-1 inhibitor may maintain or improve the antiviral efficacy achieved with continuous dosing, but with less side-effects, e.g., less body weight loss. Intermittent dose administration regimens useful in the present disclosure encompass any discontinuous administration regimen that provides a therapeutically effective amount of a LINE-1 inhibitor to a subject in need thereof. Intermittent dosing regimens can use equivalent, lower, or higher doses of the LINE-1 inhibitor than would be used in continuous dosing regimens. Advantages of intermittent dose administration of a LINE-1 inhibitor include, but are not limited to, improved safety, decreased toxicity, e.g., decreased weight loss, increased exposure, increased efficacy, and/or increased subject compliance. These advantages may be realized when the LINE-1 inhibitor is administered as a single agent or when administered in combination with one or more optional therapeutic agents. On the day a LINE-1 inhibitor is scheduled to be administered to the subject, administration can occur in a single or in divided doses, e.g., once-a-day, twice-a-day, three times a day, four times a day or more. Dosing can also occur via any suitable route, e.g., orally, intravenously, or subcutaneously. In one embodiment, the LINE-1 inhibitor is administered to the subject once (QD) or twice (BID) on the day the compound is scheduled to be administered.

The phrase “in combination” as used in connection with the administration of a LINE-1 inhibitor and one or more optional therapeutic agents to a subject means that the LINE-1 inhibitor and the one or more optional therapeutic agents can be administered to the subject together, e.g., as part of a single pharmaceutical composition or formulation, or separately, e.g., as part of two or more separate pharmaceutical compositions or formulations. The phrase “in combination” as used in connection with the administration of a LINE-1 inhibitor and the one or more optional therapeutic agents to a subject is thus intended to embrace administration of the LINE-1 inhibitor and the one or more optional therapeutic agents in a sequential manner, wherein the LINE-1 inhibitor and the one or more optional therapeutic agents are administered to the subject at a different time, as well as administration concurrently, or in a substantially simultaneous manner, e.g., less than 30 minutes apart. Simultaneous administration can be accomplished, for example, by administering to the subject a single capsule having a fixed ratio of each of the LINE-1 inhibitor and the one or more optional therapeutic agents or in multiple, single capsules for each of the LINE-1 inhibitor and the one or more optional therapeutic agents. Sequential or substantially simultaneous administration of the LINE-1 inhibitor and the one or more optional therapeutic agents can be accomplished by any appropriate route including, but not limited to, oral routes, intravenous routes, subcutaneous routes, intramuscular routes, etc. The LINE-1 inhibitor and the one or more optional therapeutic agents can be administered by the same route or by different routes. For example, the one or more optional therapeutic agents and the LINE-1 inhibitor of the combination may be administered orally. Alternatively, for example, the LINE-1 inhibitor may be administered orally and the one or more optional therapeutic agents may be administered by intravenous injection. The LINE-1 inhibitor and the one or more optional therapeutic agents may also be administered in alternation. In one embodiment, the LINE-1 inhibitor and the one or more optional therapeutic agents are administered to a subject separately, e.g., as part of two or more separate pharmaceutical compositions or formulations.

### IV. Particular Embodiments

The disclosure provides the following particular embodiments.

Embodiment 1. A method of treating or preventing a CNS or systemic disease in a subject in need thereof, the method comprising administering to the subject a therapeutically effective amount of a LINE-1 inhibitor, or a pharmaceutical composition thereof, wherein the CNS disease is ataxia-telangiectasia and the systemic disease is age-related macular degeneration, systemic lupus erythematosus, IFN-associated autoimmune disease, e.g., psoriasis, Fanconi Anemia, idiopathic pulmonary fibrosis, or cardiovascular disease, e.g., coronary heart disease.

Embodiment 2. The method of Embodiment 1 for treating or preventing ataxia-telangiectasia.

Embodiment 3. The method of Embodiment 1 for treating or preventing age-related macular degeneration.

Embodiment 4. The method of Embodiment 1 for treating or preventing systemic lupus erythematosus.

Embodiment 5. The method of Embodiment 1 for treating or preventing IFN-associated autoimmune disease, e.g., rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis.

Embodiment 6. The method of Embodiment 5 for treating or preventing psoriasis.

Embodiment 7. The method of Embodiment 1 for treating or preventing Fanconi Anemia.

Embodiment 8. The method of Embodiment 1 for treating or preventing idiopathic pulmonary fibrosis.

Embodiment 9. The method of Embodiment 1 for treating or preventing cardiovascular disease.

Embodiment 10. The method of Embodiment 9 for treating or preventing coronary heart disease.

Embodiment 11. The method of any one of Embodiments 1-10 to treat the disease.

Embodiment 12: The method of any one of Embodiments 1-10 to prevent the disease.

Embodiment 13. The method of any one of Embodiments 1-12, wherein the LINE-1 inhibitor is islatravir, censavudine, or elvucitabine.

Embodiment 14. The method of Embodiment 13, wherein the LINE-1 inhibitor is islatravir.

Embodiment 15. The method of Embodiment 13, wherein the LINE-1 inhibitor is censavudine.

Embodiment 16. The method of Embodiment 13, wherein the LINE-1 inhibitor is elvucitabine.

Embodiment 17. The method of any one of Embodiments 1-12, wherein the LINE-1 inhibitor is a compound of Formula I, or a pharmaceutically acceptable salt or solvate thereof, or a tautomer thereof.

Embodiment 18. The method of any one of Embodiments 1-12, wherein the LINE-1 inhibitor is a compound of Formula II, or a pharmaceutically acceptable salt or solvate thereof, or a tautomer thereof.

Embodiment 19. The method of any one of Embodiments 1-12, wherein the LINE-1 inhibitor is a compound of Formula III, or a pharmaceutically acceptable salt or solvate thereof, or a tautomer thereof.

Embodiment 20. The method of any one of Embodiments 1-12, wherein the LINE-1 inhibitor is a compound of Table 1, or a pharmaceutically acceptable salt or solvate thereof, or a tautomer thereof.

Embodiment 21. The method of any one of Embodiments 1-12, wherein the LINE-1 inhibitor is a compound of Table 2, or a pharmaceutically acceptable salt or solvate thereof, or a tautomer thereof.

Embodiment 22. The method of any one of Embodiments 1-21 further comprising administering one or more optional therapeutic agents to the subject.

Embodiment 23. The method of any one of Embodiments 1-22, wherein the LINE-1 inhibitor is administered orally to the subject.

Embodiment 24. A kit comprising a LINE-1 inhibitor, or a pharmaceutical composition thereof, and instructions for administering the LINE-1 inhibitor, or a pharmaceutical composition thereof, to a subject having a CNS or systemic disease.

Embodiment 25. The kit of Embodiment 24 for treating or preventing ataxia-telangiectasia.

Embodiment 26. The kit of embodiment 24 for treating or preventing age-related macular degeneration.

Embodiment 27. The kit of Embodiment 24 for treating or preventing systemic lupus erythematosus.

Embodiment 28. The kit of Embodiment 24 for treating or preventing IFN-associated autoimmune disease, e.g., rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis.

Embodiment 29. The kit of Embodiment 28 for treating or preventing psoriasis.

Embodiment 30. The kit of Embodiment 24 for treating or preventing Fanconi Anemia.

Embodiment 31. The kit of Embodiment 24 for treating or preventing idiopathic pulmonary fibrosis.

Embodiment 32. The kit of Embodiment 24 for treating or preventing cardiovascular disease.

Embodiment 33. The kit of any one of Embodiments 24-32, wherein the LINE-1 inhibitor is islatravir, censavudine, or elvucitabine.

Embodiment 34. The kit of Embodiment 33, wherein the LINE-1 inhibitor is islatravir.

Embodiment 35. The kit of Embodiment 33, wherein the LINE-1 inhibitor is censavudine.

Embodiment 36. The kit of Embodiment 33, wherein the LINE-1 inhibitor is elvucitabine.

Embodiment 37. The kit of any one of Embodiments 24-32, wherein the LINE-1 inhibitor is a compound of Formula I, or a pharmaceutically acceptable salt or solvate thereof, or a tautomer thereof.

Embodiment 38. The kit of any one of Embodiments 24-32, wherein the LINE-1 inhibitor is a compound of Formula II, or a pharmaceutically acceptable salt or solvate thereof, or a tautomer thereof.

Embodiment 39. The kit of any one of Embodiments 24-32, wherein the LINE-1 inhibitor is a compound of Formula III, or a pharmaceutically acceptable salt or solvate thereof, or a tautomer thereof.

Embodiment 40. The kit of any one of Embodiments 24-32, wherein the LINE-1 inhibitor is a compound of Table 1, or a pharmaceutically acceptable salt or solvate thereof, or a tautomer thereof.

Embodiment 41. The kit of any one of Embodiments 24-32, wherein the LINE-1 inhibitor is a compound of Table 2, or a pharmaceutically acceptable salt or solvate thereof, or a tautomer thereof.

Embodiment 42. The kit of any one of Embodiments 24-41 further comprising one or more optional therapeutic agents.

Embodiment 43. A LINE-1 inhibitor, or a pharmaceutical composition thereof, for use in treating or preventing a disease in a subject in need thereof, the method comprising administering to the subject a therapeutically effective amount of the LINE-1 inhibitor, or a pharmaceutical composition thereof, to the subject wherein the disease is ataxia-telangiectasia, age-related macular degeneration, systemic lupus erythematosus, IFN-associated autoimmune disease, e.g., psoriasis, Fanconi Anemia, idiopathic pulmonary fibrosis, or cardiovascular disease in a subject.

Embodiment 44. The LINE-1 inhibitor, or a pharmaceutical composition thereof, for use of Embodiment 43, wherein the disease is ataxia-telangiectasia.

Embodiment 45. The LINE-1 inhibitor, or a pharmaceutical composition thereof, for use of Embodiment 43, wherein the disease is age-related macular degeneration.

Embodiment 46. The LINE-1 inhibitor, or a pharmaceutical composition thereof, for use of Embodiment 43, wherein the disease is systemic lupus erythematosus.

Embodiment 47. The LINE-1 inhibitor, or a pharmaceutical composition thereof, for use of Embodiment 43, wherein the disease is IFN-associated autoimmune disease, e.g., rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis.

Embodiment 48. The LINE-1 inhibitor, or a pharmaceutical composition thereof, for use of Embodiment 47, wherein the disease is psoriasis.

Embodiment 49. The LINE-1 inhibitor, or a pharmaceutical composition thereof, for use of Embodiment 43, wherein the disease is Fanconi Anemia.

Embodiment 50. The LINE-1 inhibitor, or a pharmaceutical composition thereof, for use of Embodiment 47, wherein the disease is idiopathic pulmonary fibrosis.

Embodiment 51. The LINE-1 inhibitor, or a pharmaceutical composition thereof, for use of Embodiment 47, wherein the disease is cardiovascular disease.

Embodiment 52. The LINE-1 inhibitor, or a pharmaceutical composition thereof, for use of Embodiment 51, wherein the disease is coronary heart disease.

Embodiment 53. The LINE-1 inhibitor, or a pharmaceutical composition thereof, for use of any one of Embodiments 47-52 to treat the disease.

Embodiment 54. The LINE-1 inhibitor, or a pharmaceutical composition thereof, for use of any one of Embodiments 47-52 to prevent the disease.

Embodiment 55. The LINE-1 inhibitor, or a pharmaceutical composition thereof, for use of any one of Embodiments 47-54, wherein the LINE-1 inhibitor is islatravir, censavudine, or elvucitabine.

Embodiment 56. The LINE-1 inhibitor, or a pharmaceutical composition thereof, for use of Embodiment 55, wherein the LINE-1 inhibitor is islatravir.

Embodiment 57. The LINE-1 inhibitor, or a pharmaceutical composition thereof, of Embodiment 55, wherein the LINE-1 inhibitor is censavudine.

Embodiment 58. The LINE-1 inhibitor, or a pharmaceutical composition thereof, for use of Embodiment 55, wherein the LINE-1 inhibitor is elvucitabine.

Embodiment 59. The LINE-1 inhibitor of any one of Embodiments 47-54, wherein the LINE-1 inhibitor is a compound of Formula I, or a pharmaceutically acceptable salt or solvate thereof, or a tautomer thereof.

Embodiment 60. The LINE-1 inhibitor of any one of Embodiments 47-54, wherein the LINE-1 inhibitor is a compound of Formula II, or a pharmaceutically acceptable salt or solvate thereof, or a tautomer thereof.

Embodiment 61. The LINE-1 inhibitor of any one of Embodiments 47-54, wherein the LINE-1 inhibitor is a compound of Formula III, or a pharmaceutically acceptable salt or solvate thereof, or a tautomer thereof.

Embodiment 62. The LINE-1 inhibitor of any one of Embodiments 47-54, wherein the LINE-1 inhibitor is a compound of Table 1, or a pharmaceutically acceptable salt or solvate thereof, or a tautomer thereof.

Embodiment 63. The LINE-1 inhibitor of any one of Embodiments 47-54, wherein the LINE-1 inhibitor is a compound of Table 2, or a pharmaceutically acceptable salt or solvate thereof, or a tautomer thereof.

Embodiment 64. The LINE-1 inhibitor, or a pharmaceutical composition thereof, for use of any one of Embodiments 47-63, wherein the LINE-1 inhibitor, or a pharmaceutical composition thereof, is to be administered to the subject with one or more optional therapeutic agents.

Embodiment 65. The LINE-1 inhibitor, or a pharmaceutical composition thereof, for use of any one of Embodiments 47-64, wherein the LINE-1 inhibitor is to be administered orally to the subject.

Embodiment 66. Use of a LINE-1 inhibitor, or a pharmaceutical composition thereof, in the manufacture of a medicament in treating or preventing a disease in a subject, wherein the disease is ataxia-telangiectasia, age-related macular degeneration, systemic lupus erythematosus, IFN-associated autoimmune disease, e.g., psoriasis, Fanconi Anemia, idiopathic pulmonary fibrosis, or cardiovascular disease.

Embodiment 67. The use of Embodiment 66, wherein the disease is ataxia-telangiectasia.

Embodiment 68. The use of Embodiment 66, wherein the disease is age-related macular degeneration.

Embodiment 69. The use of Embodiment 66, wherein the disease is systemic lupus erythematosus.

Embodiment 70. The use of Embodiment 66, wherein the disease is IFN-associated autoimmune disease, e.g., rheumatoid arthritis, psoriasis, vitiligo, hypothyroidism, hyperthyroidism, idiopathic thrombocytopenic purpura, autoimmune hemolytic anemia, myasthenia gravis, Addison disease, celiac disease, polymyositis, or superimposed autoimmune hepatitis.

Embodiment 71. The use of Embodiment 70, wherein the disease is psoriasis.

Embodiment 72. The use of Embodiment 66, wherein the disease is Fanconi Anemia.

Embodiment 73. The use of Embodiment 66, wherein the disease is idiopathic pulmonary fibrosis.

Embodiment 74. The use of Embodiment 66, wherein the disease is cardiovascular disease.

Embodiment 75. The use of Embodiment 74, wherein the disease is coronary heart disease.

Embodiment 76. The use of any one of Embodiments 66-76 to treat the disease.

Embodiment 77: The use of any one of Embodiments 66-76 to prevent the disease.

Embodiment 78. The use of any one of Embodiments 66-77, wherein the LINE-1 inhibitor is islatravir, censavudine, or elvucitabine.

Embodiment 79. The use of Embodiment 78, wherein the LINE-1 inhibitor is islatravir.

Embodiment 80. The use of Embodiment 78, wherein the LINE-1 inhibitor is censavudine.

Embodiment 81. The use of Embodiment 78, wherein the LINE-1 inhibitor is elvucitabine.

Embodiment 82. The use of any one of Embodiments 66-77, wherein the LINE-1 inhibitor is a compound of Formula I, or a pharmaceutically acceptable salt or solvate thereof, or a tautomer thereof.

Embodiment 83. The use of any one of Embodiments 66-77, wherein the LINE-1 inhibitor is a compound of Formula II, or a pharmaceutically acceptable salt or solvate thereof, or a tautomer thereof.

Embodiment 84. The use of any one of Embodiments 66-77, wherein the LINE-1 inhibitor is a compound of Formula III, or a pharmaceutically acceptable salt or solvate thereof, or a tautomer thereof.

Embodiment 85. The use of any one of Embodiments 66-77, wherein the LINE-1 inhibitor is a compound of Table 1, or a pharmaceutically acceptable salt or solvate thereof, or a tautomer thereof.

Embodiment 86. The use of any one of Embodiments 66-77, wherein the LINE-1 inhibitor is a compound of Table 2, or a pharmaceutically acceptable salt or solvate thereof, or a tautomer thereof.

Embodiment 87. The use of any one of Embodiments 66-86 further comprising administering one or more optional therapeutic agents to the subject.

Embodiment 88. The use of any one of Embodiments 66-87, wherein the LINE-1 inhibitor, or a pharmaceutical composition thereof, is to be administered orally to the subject.

Embodiment 89. The method of any one of Embodiments 1-23, wherein the subject is (a) not infected with the HIV virus, (b) is not suspected of being infected with the HIV virus, and/or (c) is not being treated to prevent infection with the HIV virus.

Embodiment 90. The kit of any one of Embodiments 24-42, wherein the subject is (a) not infected with the HIV virus, (b) is not suspected of being infected with the HIV virus, and/or (c) is not being treated to prevent infection with the HIV virus.

Embodiment 91. The LINE-1 inhibitor, or a pharmaceutical composition thereof, for use of any one of Embodiments 43-65, wherein the subject is (a) not infected with the HIV virus, (b) is not suspected of being infected with the HIV virus, and/or (c) is not being treated to prevent infection with the HIV virus.

Embodiment 92. The use of any one of Embodiments 66-88, wherein the subject is (a) not infected with the HIV virus, (b) is not suspected of being infected with the HIV virus, and/or (c) is not being treated to prevent infection with the HIV virus.

The disclosure also provides the following particular embodiments.

Embodiment I. A method of treating or preventing a disease in a subject in need thereof, the method comprising administering to the subject a therapeutically effective amount of a LINE-1 inhibitor, or a pharmaceutical composition thereof, wherein the disease is ataxia-telangiectasia, age-related macular degeneration, systemic lupus erythematosus, IFN-associated autoimmune disease, Fanconi Anemia, idiopathic pulmonary fibrosis, or cardiovascular disease.

Embodiment II. The method of Embodiment I, wherein the disease is ataxia-telangiectasia.

Embodiment III. The method of Embodiment I, wherein the disease is age-related macular degeneration.

Embodiment IV. The method of Embodiment I, wherein the disease is systemic lupus erythematosus.

Embodiment V. The method of Embodiment I, wherein the disease is IFN-associated autoimmune disease.

Embodiment VI. The method of Embodiment V, wherein the disease is psoriasis.

Embodiment VII. The method of Embodiment I, wherein the disease is Fanconi Anemia.

Embodiment VIII. The method of Embodiment I, wherein the disease is idiopathic pulmonary fibrosis.

Embodiment IX. The method of Embodiment I, wherein the disease is cardiovascular disease.

Embodiment X. The method of any one of Embodiments I-IX, wherein the LINE-1 inhibitor is islatravir, censavudine, or elvucitabine.

Embodiment XI. The method of any one of Embodiments I-X, wherein the LINE-1 inhibitor is islatravir.

Embodiment XII. The method of Embodiment XI, wherein about 0.1 mg to about 10 mg of islatravir is orally administered to the subject per day.

Embodiment XIII. The method of any one of Embodiments I-X, wherein the LINE-1 inhibitor is censavudine.

Embodiment XIV. The method of any one of Embodiments I-X, wherein the LINE-1 inhibitor is elvucitabine.

Embodiment XV. The method of any one of Embodiments I-XIV for treating the disease.

Embodiment XVI. The method of any one of Embodiments I-XIV for preventing the disease.

Embodiment XVII. The method of any one of Embodiments I-XVI, further comprising administering one or more optional therapeutic agents to the subject.

Embodiment XVIII. The method of any one of Embodiments I-XVII, wherein the subject is a human.

Embodiment XIX. The method of any one of Embodiments I-XVIII, wherein the subject is (a) not infected with the HIV virus, (b) is not suspected of being infected with the HIV virus, and/or (c) is not being treated to prevent infection with the HIV virus.

Embodiment XX. A kit comprising a LINE-1 inhibitor, or a pharmaceutical composition thereof, and instructions of administering the LINE-1 inhibitor, or pharmaceutical composition thereof, to a subject having a disease, wherein the disease is ataxia-telangiectasia and the systemic disease is age-related macular degeneration, systemic lupus erythematosus, IFN-associated autoimmune disease, Fanconi Anemia, idiopathic pulmonary fibrosis, or cardiovascular disease.

Embodiment XXI. The kit of Embodiment XX, wherein the LINE-1 inhibitor is islatravir, censavudine, or elvucitabine.

Embodiment XXII. The kit of Embodiment XXI, wherein the LINE-1 inhibitor is islatravir.

Embodiment XXIII. The kit of Embodiment XXI, wherein the LINE-1 inhibitor is censavudine.

Embodiment XXIV. The kit of Embodiment XXI, wherein the LINE-1 inhibitor is elvucitabine.

Embodiment XXV. The kit of any one of Embodiments XX-XXIV further comprising one or more optional therapeutic agents.

Embodiment XXVI. The kit of any one of Embodiments XX-XXV, wherein the subject is (a) not infected with the HIV virus, (b) is not suspected of being infected with the HIV virus, and/or (c) is not being treated to prevent infection with the HIV virus.

### EXAMPLES

### Example 1

### Human LINE-1 Retrotransposition Assay

Islatravir and other compounds were tested for inhibition of retrotransposition activity of human LINE-1 in HeLa cells according to the following procedure.

HeLa cervical cancer cells were cultivated at 37° C. in a humidified 5% CO₂ incubator in Dulbecco's Modified Eagle's Medium (DMEM)—high glucose, with 4500 mg/L glucose, L-glutamine, sodium pyruvate and sodium bicarbonate (Sigma), supplemented with 10% of heat inactivated fetal bovine serum (Thermo Fisher).

Assays were performed using reporter plasmid pYX017 as described (Xie, et al., 2011) with several modifications. The reporter assay was performed in 96-well white optical bottom plates. HeLa cells were seeded in wells 24 h prior to transfection and compound treatment so that cells were approximately 30% confluent on the day of transfection. Different cell plating densities were tested and a density of 2×10³ cells was determined to be optimal.

Compounds were resuspended in DMSO. Serial dilutions (1:3) were prepared in DMSO. Medium containing different concentrations of the compounds were prepared by adding 2 μl of the compound dilution to 1 ml of the culture medium. The final concentration of DMSO in the medium was 0.2%.

FuGENE® HD transfection reagent (Promega, E2311, Lot 382574 and Lot 397842) was used to transfect the plasmids into the cells. The transfection reagent: DNA mixture was prepared in OpiMEM (Thermo Fisher) according to manufacturer's instructions. Different ratios of transfection reagent to DNA were tested and a ratio of 3:1 was determined to be optimal. Culture medium was removed from the cells and discarded. The transfection reagent: DNA mixture (5 μl) was mixed with the compound containing medium (100 μl/well) and this was added onto the cells of each well. Cells were incubated at 37° C./5% CO₂ for different incubation time. A 72 h incubation time was determined to be optimal.

Luciferase reporter activity was quantified with the Dual-Luciferase® Reporter Assay System (Promega) according to manufacturer's instructions for multiwell plates except that cells were lysed directly on the multiwell plate with 30 μl of the passive lysis buffer (PLB) for 20 min at room temperature, with gentle shaking to ensure complete cell lysis.

Firefly and Renilla luciferase signals were measured using a SpectraMax i3x Multi-Mode Microplate Reader. Integration times of 100 ms and 10 ms were used to measure the Firefly and Renilla signals respectively. Relative L1 activity is calculated as Firefly/Renilla *1000 or Firefly/Renilla *10,000. Dose response inhibition data were fit to a four parameter logistic equation using non-linear regression (using Graphpad Prism 8), to determine IC₅₀ values for each inhibitor.

The results are provided in Table 3 and Table 4. Islatravir and censavudine exhibited unexpectedly better human LINE-1 inhibitor activity compared to the other reverse transcriptase inhibitory drugs tested.

TABLE 3
Human L1 activity inhibition

| | Human LINE-1 | | | | |
|---|---|---|---|---|---|
| | IC₅₀ (μM), n = 2-4 | | | | |
| Compound | Exp. 1 | Exp. 2 | Exp. 3 | Exp. 4 | Average |
| Lamivudine | 0.79 | 0.87 | 0.57 | 0.34 | 0.64 |
| Censavudine | 0.05 | 0.09 | 0.08 | 0.07 | 0.07 |
| Bictegravir | 12.41 | >50 | N/A | — | >12.41 |
| Efavirenz | >50 | >0.25 | N/A | — | >0.25 |
| Nevirapine | >50 | >50 | N/A | — | >50 |
| Rilpivirine | >50 | 15.08 | 45.49 | — | >15.08 |
| Zidovudine | 0.41 | 0.34 | 1.68 | — | 0.81 |
| Islatravir | — | 0.00118 | 0.00140 | — | 0.0013 |
| Raltegravir | 9.86 | >50 | 49.28 | — | >9.86 |
| potassium | | | | | |
| Dolutegravir | 9.38 | N/A | >20 | — | >9.38 |
| sodium | | | | | |
| Emtricitabine | — | — | 1.39 | 1.30 | 1.34 |
| Apricitabine | — | — | 7.69 | 5.22 | 6.46 |
| Tenofovir | — | — | 0.14 | 0.26 | 0.2 |
| disoproxil | | | | | |
| Tenofovir | — | — | 3.31 | 2.23 | 2.77 |
| Elvucitabine | 0.09 | — | 0.08 | 0.11 | 0.09 |
| Abacavir sulfate | — | — | 16.77 | 17.44 | 17.10 |
| Stavudine | — | — | 0.78 | 0.71 | 0.75 |

TABLE 4
Human L1 activity inhibition

| Cpd. No. | Human LINE-1 |
|---|---|
| (of Table 1) | IC₅₀ (μM) |
| 2 | 0.39 |
| 4 | 0.49 |
| 6 | 18.6 |
| 11 | >25 |
| 12 | 0.91 |
| 13 | 12.5 |
| 14 | >12.5 |
| 15 | 0.011 |
| 17 | >50 |
| 18 | 0.008 |
| 19 | 2.0 |
| 20 | 0.003 |

Having now fully described the compounds, methods, kits, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications and publications cited herein are fully incorporated by reference herein in their entirety.

1. A method of treating or preventing a disease in a subject in need thereof, the method comprising administering to the subject a therapeutically effective amount of a LINE-1 inhibitor, or a pharmaceutical composition thereof, wherein the disease is ataxia-telangiectasia, age-related macular degeneration, systemic lupus erythematosus, IFN-associated autoimmune disease, Fanconi Anemia, idiopathic pulmonary fibrosis, or cardiovascular disease, and the LINE-1 inhibitor is censavudine,

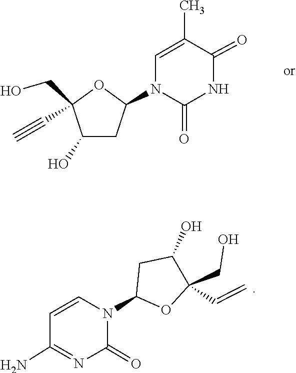 [SMILES: C#C[C@]1(CO)O[C@@H](n2cc(C)c(=O)[nH]c2=O)C[C@@H]1O ; C=C[C@]1(CO)O[C@@H](n2ccc(N)nc2=O)C[C@@H]1O]

2. The method of claim 1, wherein the disease is ataxia-telangiectasia.

3. The method of claim 1, wherein the disease is age-related macular degeneration.

4. The method of claim 1, wherein the disease is systemic lupus erythematosus.

5. The method of claim 1, wherein the disease is IFN-associated autoimmune disease.

6. The method of claim 5, wherein the disease is psoriasis.

7. The method of claim 1, wherein the disease is Fanconi Anemia.

8. The method of claim 1, wherein the disease is idiopathic pulmonary fibrosis.

9. The method of claim 1, wherein the disease is cardiovascular disease.

10-12. (canceled)

13. The method of claim 1, wherein the LINE-1 inhibitor is censavudine.

14-16. (canceled)

17. The method of claim 1 for treating the disease.

18. The method of claim 1 for preventing the disease.

19. The method of claim 1, further comprising administering one or more optional therapeutic agents to the subject.

20. (canceled)

21. The method of claim 1, wherein the subject is (a) not infected with the HIV virus, (b) is not suspected of being infected with the HIV virus, and/or (c) is not being treated to prevent infection with the HIV virus.

22. A kit comprising a LINE-1 inhibitor, or a pharmaceutical composition thereof, and instructions of administering the LINE-1 inhibitor, or pharmaceutical composition thereof, to a subject having a disease, wherein the disease is ataxia-telangiectasia, age-related macular degeneration, systemic lupus erythematosus, IFN-associated autoimmune disease, Fanconi Anemia, idiopathic pulmonary fibrosis, or cardiovascular disease, and the LINE-1 inhibitor is censavudine,

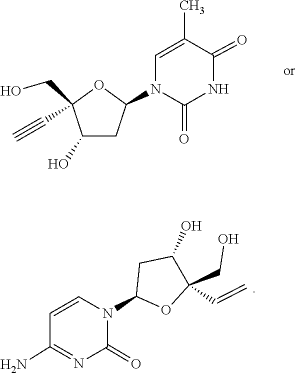 [SMILES: C#C[C@]1(CO)O[C@@H](n2cc(C)c(=O)[nH]c2=O)C[C@@H]1O ; C=C[C@]1(CO)O[C@@H](n2ccc(N)nc2=O)C[C@@H]1O]

23. (canceled)

24. (canceled)

25. The kit of claim 22, wherein the LINE-1 inhibitor is censavudine.

26-28. (canceled)

29. The kit of claim 22 further comprising one or more optional therapeutic agents.

30. The kit of claim 22, wherein the subject is (a) not infected with the HIV virus, (b) is not suspected of being infected with the HIV virus, and/or (c) is not being treated to prevent infection with the HIV virus.

31. The method of claim 1, wherein the LINE-1 inhibitor is:

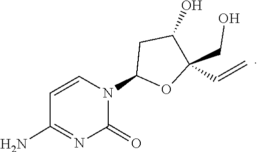 [SMILES: C=C[C@]1(CO)O[C@@H](n2ccc(N)nc2=O)C[C@@H]1O]

32. The method of claim 22, wherein the LINE-1 inhibitor is: 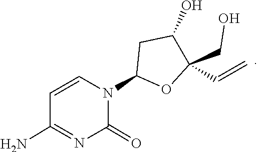 [SMILES: C=C[C@]1(CO)O[C@@H](n2ccc(N)nc2=O)C[C@@H]1O]
